(19) United States
(12) Patent Application Publication
(10) Pub. No.: US 2016/0007888 A1
(43) Pub. Date: Jan. 14, 2016

(54) Wearable activity monitoring device and related method

(71) Applicant: Suunto Oy, Vantaa (FI)

(72) Inventors: Heikki Nieminen, Vantaa (FI); Mikko Martikka, Vantaa (FI); Markus Kemetter, Helsinki (FI); Michael Miettinen, Vantaa (FI); Erik Lindman, Vantaa (FI)

(21) Appl. No.: 14/331,268

(22) Filed: Jul. 15, 2014

(30) Foreign Application Priority Data
Jul. 11, 2014 (FI) 20145671

(51) Int. Cl. A61B 5/11; A61B 5/00

(52) U.S. Cl. CPC A61B 5/1118; A61B 5/1123; A61B 5/7246; A61B 5/0022; A61B 5/681

(57) ABSTRACT

The invention relates to a device and method for monitoring physical activity of a person. The device comprises an acceleration sensor for providing acceleration data corresponding to movements of the person, a memory unit capable of storing an activity index value describing cumulative physical activity of the person, and data processing unit capable of being operated in at least a first activity monitoring mode in which the data processing unit is adapted to update the activity index value based on the acceleration data. In addition, the processing unit is, in the first activity monitoring mode, configured to determine if, and to update the activity index value only if, the acceleration data corresponds to walking- and/or running-related movements of the person. The invention allows for robust monitoring of activities that have an impact on fitness and health of individuals and reduce the risk of incorrect activity recordings.

### FIELD OF THE INVENTION

The invention relates to wearable electronic devices for monitoring physical activities of a person. The invention also concerns related methods for activity tracking.

### BACKGROUND OF THE INVENTION

Wearable activity monitoring devices, so-called “activity trackers” are intended to determine overall activity of a person during his/her daily duties and have become popular lately. There are devices utilizing heart rate measurement, acceleration measurement and satellite positioning systems. Some devices utilize only internal sensors, some receive data from external modules, such as foot pods, heart rate sensors or positioning pods. There are devices that are provided in the form of modules attachable to a chest band or other piece of garment or body part. One class of devices is provided in the form of wristbands (so-called “activity bracelets)” with integral accelerometer for measurement of movements of the users wrist during his or her daily activities and determination of an activity index, which is presented to the user. The activity tracking can also be implemented as a function in a multifunction digital wrist watch, wristop computer or other mobile device, such as a mobile phone.

In particular for people aiming at serious activity tracking for the purposes of fitness monitoring or body weight control, the activity indices given by the existing devices, in particular those based on acceleration only, are often too inaccurate or inconsistent between different usage periods. There is a particular difficulty in determining activity using acceleration sensors located on the wrist of the user, because wrist movement can relate either to ordinary lightweight daily tasks or hard-intensity training, or anything between these extremes. Therefore, as concerns the real physiological effect of the activities done, either too little or too much activity may be registered.

For obtaining a better estimate of real activity, one can try to detect which kind of exercise the user is carrying out. U.S. Pat. No. 7,328,612 discloses a method and apparatus for detecting types of exercise from an acceleration signal using at least two different characteristics computed from the acceleration signal and comparison tables for the characteristics stored in the memory of the apparatus. The method is particularly designed for acceleration signal measured at a lower limb of a person and is not expected to provide equally reliable results for wrist-based measurements, because wrist movement is only loosely related to feet movements. U.S. Pat. No. 5,989,200 discloses a method which computes and utilizes an average of peak accelerations caused by steps.

One further both technical and user-experience-related problem of the presently available activity trackers is a short battery life.

Thus, there is a need for improved activity tracking devices and methods.

### SUMMARY OF THE INVENTION

It is an aim of the invention to provide a novel activity monitoring device overcoming at least some of the abovementioned problems and in particular being capable of giving physiologically more accurate activity estimates. A further aim is to provide a corresponding activity monitoring method.

The invention is based on the general idea of drawing conclusions on the activity of the person based only on such activities that can be reliably detected, mainly walking and running, in a background monitoring mode.

According to a first aspect, the invention provides a device for monitoring physical activity of a person, comprising an acceleration sensor for providing acceleration data corresponding to movements of the person, a memory unit capable of storing an activity index value describing a cumulative physical activity of the person, and a data processing unit capable of being operated in at least a first activity monitoring mode in which the data processing unit is adapted to update the activity index value based on the acceleration data. In addition, the processing unit is in the first activity monitoring mode configured to determine if, and to update the activity index value only if, the acceleration data corresponds to walking- and/or running-related movements of the person.

According to a second aspect, the device comprises an acceleration sensor and memory unit as described above and a data processing unit adapted to update the activity index value based on the acceleration data according to update criteria. The data processing unit is capable of being operated in at least a first activity monitoring mode and a second activity monitoring mode and is configured to use different update criteria in the first and second activity monitoring modes.

According to a third aspect, the invention provides a device for monitoring physical activity of a person, comprising an acceleration sensor as described above and a data processing unit for analyzing the acceleration data for computing an activity index describing a cumulative activity of the person, the data processing unit comprising means for detecting periodic motions from the acceleration data, means for determining frequency of the periodic motions, and means for determining whether the frequency of the periodic motions is within a predefined frequency range. The data processing unit further comprises means for determining the average acceleration based on the acceleration data, means for determining whether the average acceleration is within a predefined acceleration range, and means for updating the activity index using the frequency of periodic motions and average acceleration, if the frequency of the periodic motions and the average acceleration are within the predefined ranges.

The present method for monitoring physical activity of a person in a wearable device, comprises providing acceleration data corresponding to wrist movements of the person, and analyzing the acceleration data unit in a first activity monitoring mode of the wearable device in order to derive an activity index corresponding to intensity of the physical activity by detecting features of the acceleration data that correspond to cadence of walking and/or running, and if such features are found, updating the activity index using these features.

More specifically, the invention is characterized by what is stated in the independent claims.

The invention provides considerable advantages. The invention provides an efficient activity monitoring device that can operate “in the background”, without the need of the user to specifically switch to a training mode. Compared with conventional activity monitoring devices, the present device concentrates on detecting and recording activity of walking and running, which are the most common types of everyday exercises that are beneficial for individuals. They typically form a major portion of physical performances of an average person that has a positive effect on the fitness of the person, in addition to specific training sessions the person is doing (e.g. gym training or team sports). Walking and running can be reliably distinguished from other activities by means of embodiments of the invention and the physical intensity, i.e., load for the body, of walking and running can be reliably determined using the acceleration data measured from wrist. Thus, a very reliable estimate on the effect of these activities on the metabolic system of a person is obtained. The risk of coarse misestimates is reduced, since arbitrary accelerations, which cannot be associated with walking or running, are not used.

In particular, the present device may be configured to detect and record at least walking activity in the first activity monitoring mode. Walking typically forms a majority of everyday activities that are beneficial for health and the cumulative amount of walking in the form of the activity index is therefore of particular interest to many users. As a secondary additional function, the detection and recording of running is implemented. Running for short distances outside actual running sessions (which are preferably recorded in a separate activity monitoring mode) is also not uncommon and recording thereof according to embodiments of the invention provides additional advantages.

For other training sessions, the user may be given the option to record activity in one or more separate training modes, potentially utilizing also other sensors than the built-in acceleration sensor.

In a preferred embodiment, the present device is a multifunction digital wrist watch or wristop computer, having the present activity monitoring implemented as one function thereof. If desired, the monitoring may in principle take place “24/7”, i.e. essentially all times the device is worn, or at least during daytime.

The present device preferably utilizes in the first activity monitoring mode only the acceleration sensor and not any other sensors. This saves a considerable amount of battery energy compared with for example the use of GPS sensor or radio link with an external sensor, such as a heart rate sensor or foot or bike pod.

The present device is preferably a wrist-worn device. Compared with mobile phones having acceleration sensing capabilities, the present device has the advantage that the movement of wrist during walking and running is relatively well defined, and therefore running and walking is capable of being accurately determined by means the present invention. A traditional mobile phone cannot be conveniently attached to wrist, unlike a wristop computer, such as a sports or outdoor computer. It is also not desirable to run additional energy-consuming measurement and computing operations in mobile phones, whose operating times are low even without such operations. A mobile phone is also often left on a table, for example, whereby the persons activity is not recorded.

Compared with devices using satellite positioning to determine overall activity, the present invention has considerably lower energy consumption and also works indoors.

The dependent claims are focused on selected embodiments of the invention.

According to one embodiment, the processing unit is in the first activity monitoring mode configured to determine if the acceleration data corresponds to walking- and/or running-related movements of the person. This can be carried out by analyzing periodicity of the acceleration data to find cadence whose characteristics can be deduced to originate from walking or running. If such cadence is found, the activity index is derived using the acceleration data. This approach is in contrast with that of U.S. Pat. No. 5,989,200 discussed above, as the periodicity, i.e., frequency and temporal regularity of repeating features of the acceleration data, is used to indicate walking- or running-based cadence.

According to one embodiment, the processing unit is configured to determine if the acceleration data corresponds to walking- and/or running-related movements of the person by analyzing average magnitude of the acceleration data. This embodiment can be combined with the abovementioned one such that both the cadence and average acceleration are used to derive the activity index.

In still more detail, according to one embodiment, the data processing unit is configured to detect periodic motions from the acceleration data, to determine frequency of the periodic motions, to determine whether the frequency of the periodic motions is at a predefined frequency range, and to update (increase) the activity index value using the frequency of periodic motions only if the frequency of the periodic motions is at the predefined frequency range.

In one embodiment, the data processing unit is configured to determine average acceleration based on the acceleration data, to determine whether the average acceleration is at a predefined acceleration range, and update the activity index using the average acceleration only if the average acceleration is at the predefined acceleration range.

According to one embodiment, the processing unit is additionally configured to use at least one of the following parameters storable in the memory if the device for updating the activity index value: age of the person, sex of the person, mass of the person, fitness index of the person, maximum oxygen intake value (VO_{2,max}) of the person. These parameters can be fed to the device manually by the user through user interface.

According to one embodiment, the processing unit is configured to determine average acceleration based on the acceleration data, to convert the average acceleration determined to average speed of the person using a conversion function stored in the memory of the device, and to use the average speed for updating the activity index value. The device can also comprise means for receiving speed data of the person from a (wireless) speed sensor (e.g. satellite positioning sensor or foot pod) and to adapt properties of the conversion function in the memory of the device so as to reflect the correct dependence between the average acceleration and the speed data.

The abovementioned embodiments and those that will be described later or claimed can be combined with each other and with the different aspects of the invention.

The term “activity index” refers to any parameter value which is derived from physical movements of the person by means of measuring acceleration of the person and processing the acceleration data in a computing unit in accordance with the invention. In particular, the activity index is a cumulative, i.e. monotonically increasing value, which describes the amount of motoric or metabolic activity of the person (over basic activity) due to walking and/or running carried out by the person during a measurement period. The activity index can be reset for example automatically on a daily basis, or in response to a user command. The activity index can be a dimensionless quantity or one with a dimension. In particular, it can show a relative intensity. It is also possible to compute other parameters, such as energy consumption or time needed for full recovery from preceding physical performances (recovery time), based on the activity index. The activity index can also itself represent energy consumption or recovery time. Energy consumption has an established meaning in the art, whereas the concept of recovery time and one exemplary method for calculation thereof is described in US 2011/263993.

“Recording activity” means updating the activity index using a measurement and computing process in accordance with the invention.

“Activity monitoring mode” is an operating mode of the data processing unit of the device in which a certain acceleration measurement rules and acceleration data processing algorithms are used to record activity. The invention focuses on the implementation of a “first activity monitoring mode” which can be operated in the background during normal other daily use of the device to automatically record desired activities during the day and to neglect some other activities. However, in certain aspects and embodiments of the invention, also one or more “second activity monitoring modes” typically requiring user start/stop actions each time a particular physical exercise is about to start/stop, are implemented. The one or more second activity monitoring modes can have an essential relation to the first activity monitoring mode in that they may update the same activity index and/or be used to autocalibrate the algorithm, parameters or data conversion functions used in the first activity monitoring mode.

“Walking-related movements” of a person mean body movements (in particular wrist movements when a wrist-worn device is used) that are associated with normal human gait at the pace of walking (which is typically less than 6 km/h). In particular, “walking-related movements” are movements that produce acceleration data meeting predefined first criteria for magnitude and periodicity (frequency and stability of the frequency over a time period). Correspondingly, “running-related movements” mean movements that are associated with normal gait at the pace of running (typically 6 km/h or more). In particular, “running-related movements” are movements that produce acceleration data meeting predefined second criteria for magnitude and periodicity, the second criteria being different from the first criteria. The second criteria can be applied after application of the first criteria, thus prioritizing the detection of walking.

Next, embodiments and advantages of the invention are described in more detail with reference to the attached drawings.

### BRIEF DESCRIPTION OF THE DRAWINGS

FIG. 1 shows a schematic view of a device according to the invention.

FIG. 2 shows an exemplary graph of intensity of a persons activities over time, as well as periods of automatic and optional user-initiated activity recording using a device according to the invention.

FIGS. 3A and 3B show flow charts of the method according to selected embodiments of the invention.

FIG. 3C shows a flow chart of the implementation of the invention according to one embodiment.

FIGS. 4A and 4B show a running speed vs. average acceleration graph and acceptable average acceleration and cadence graphs which can be used by the present device to implement robust walking and running activity determination.

### DETAILED DESCRIPTION OF EMBODIMENTS

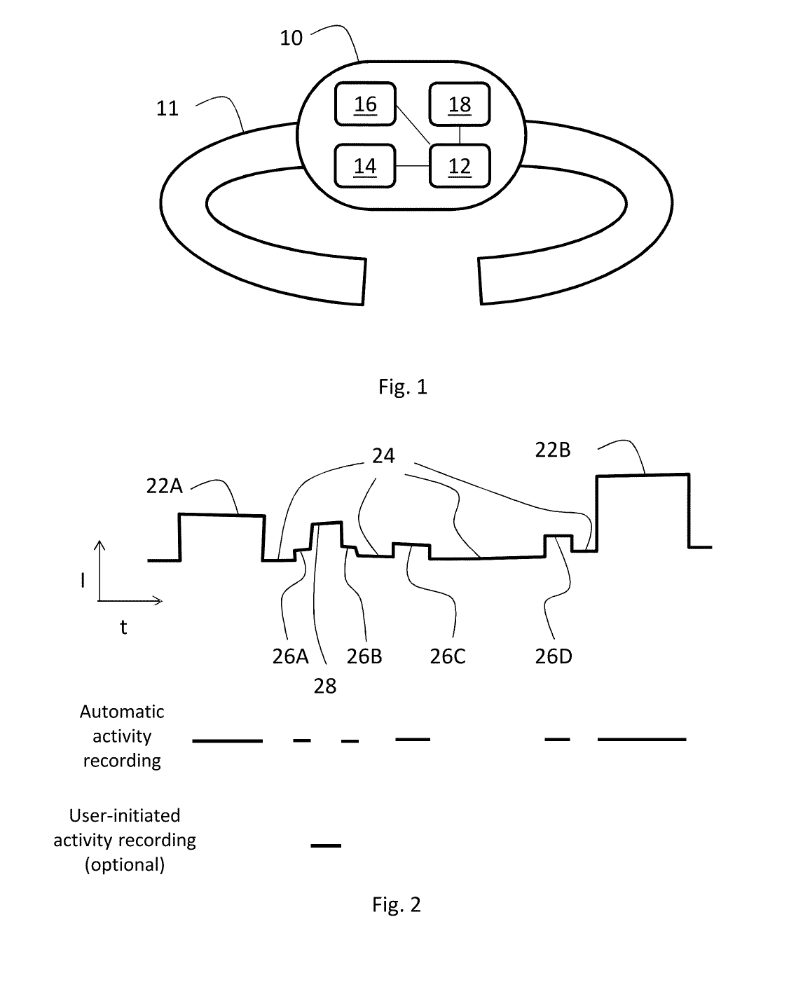

FIG. 1 shows a device according to one embodiment the invention. The device comprises a main unit 10 and a wristband 11 attached to the main unit 10. The main unit comprises a data processing unit 12 and an acceleration sensor 14, memory unit 16 and user interface unit 18 functionally connected to the data processing unit 12. The data processing unit 12 can comprise a microcontroller, optionally with the memory unit 16 built therein. The data processing unit 12 is capable of running a program code including instructions necessary for the device to operate according to the invention.

The acceleration sensor 14 can comprise a one-, two- or most preferably a three-dimensional accelerometer, which are well known in the art.

The user interface unit 18 herein comprises the necessary means for the user to interact with the program code of the device, e.g. switch on and off the device, to change mode of activity monitoring mode of the device or to input personal parameters (input means) and optionally to visually inspect the determined activity index (output means). The input means may comprise one or more buttons or a touchscreen. The output means may comprise a display of any kind. According to one embodiment, either the input means, output means or both are implemented as a contact-based or wireless communication means allowing the user to interact with the device using another device, such as mobile phone or computer, over a communication link.

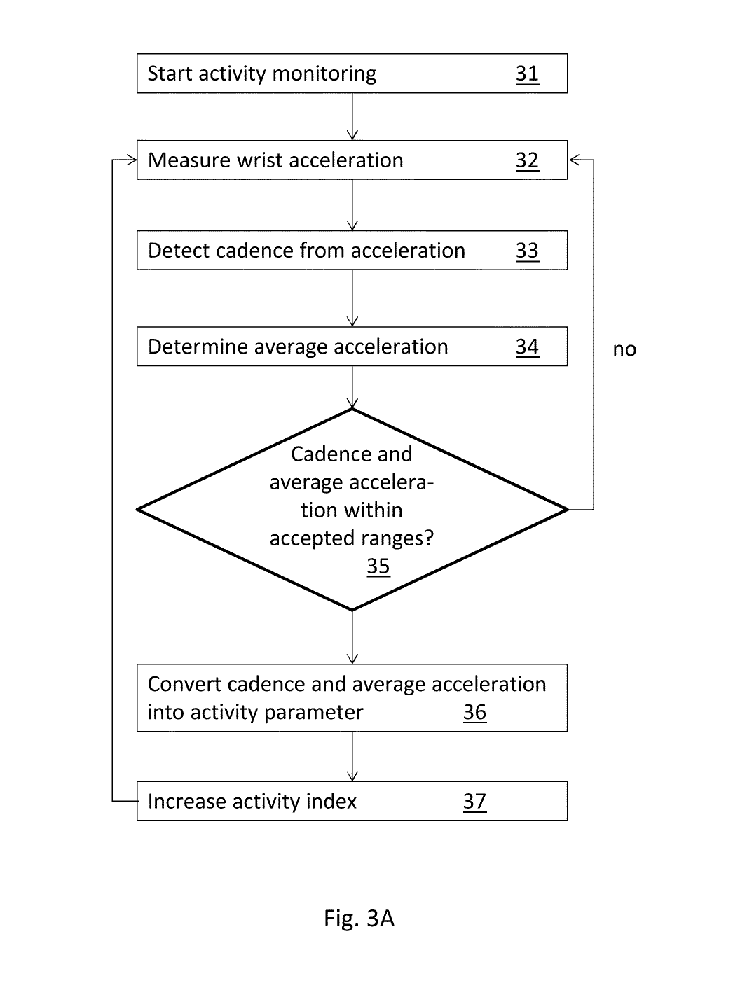

FIG. 2 shows a graph illustrating the operation of the device in an automatic activity determination mode during an exemplary time period, including different types of activities of a person that cause the person's metabolic activity to temporarily rise over a metabolic base rate. Intensity periods 22A and 22B represent running performances (for example running in the morning to a workplace and in the afternoon back to home). Intensity periods 26A-D represent walking performances during the workday. Intensity period 28 represents a non-walking and non-running performance (such as a gym training session), preceded and followed by short walks 26A and 26B (to the gym and back). Intermediate periods 24 represent everything else the person in doing during the day (such as ordinary office work with metabolic activity close to metabolic base rate).

As illustrated, the automatic activity determination is designed to detect and record activity during the running and walking periods 22A-B and 26A-D, but not during the gym (or some other sports) period 28. If the activity during the gym period is to be recorded, the device may be provided with another mode of operation which the user initiates through the interface unit of the device. This second mode of operation (“second activity monitoring mode”, discussed later in more detail) is typically not based on cadence detection and determination of the intensity of activity based on cadence, but some other algorithm better reflecting the intensity of the particular exercise into the activity index.

There may be a common activity index for all types of activities recorded, or two or more separate activity indices, which are updated separately depending on the activity monitoring mode concerned.

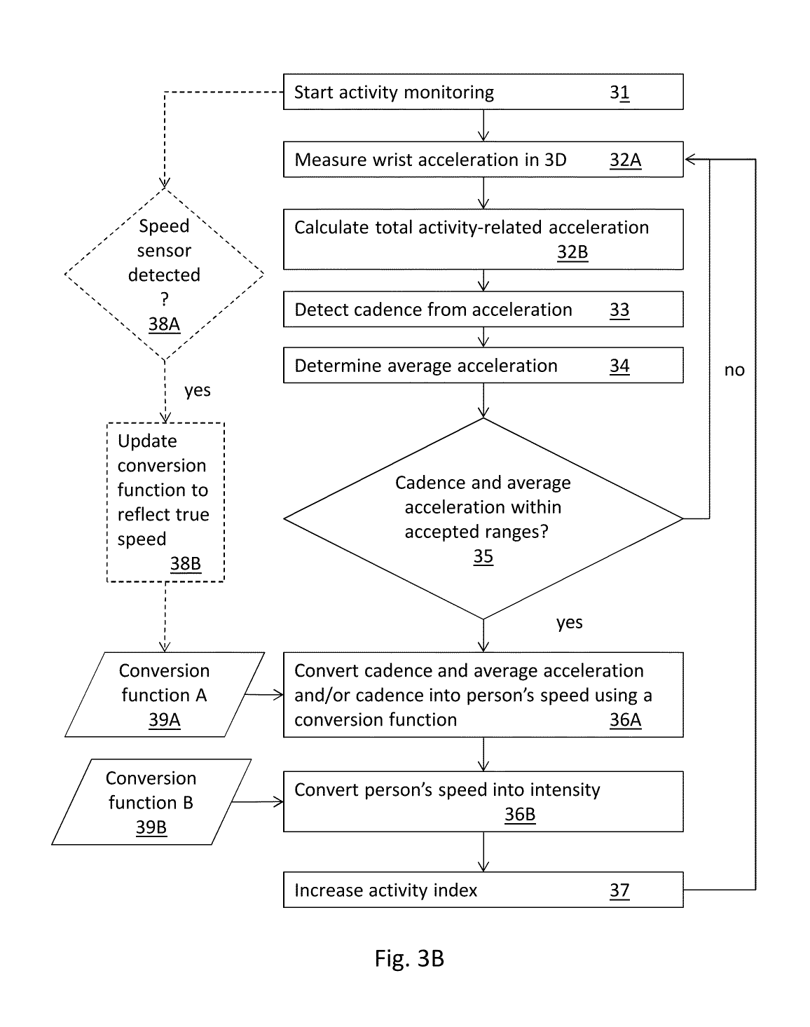

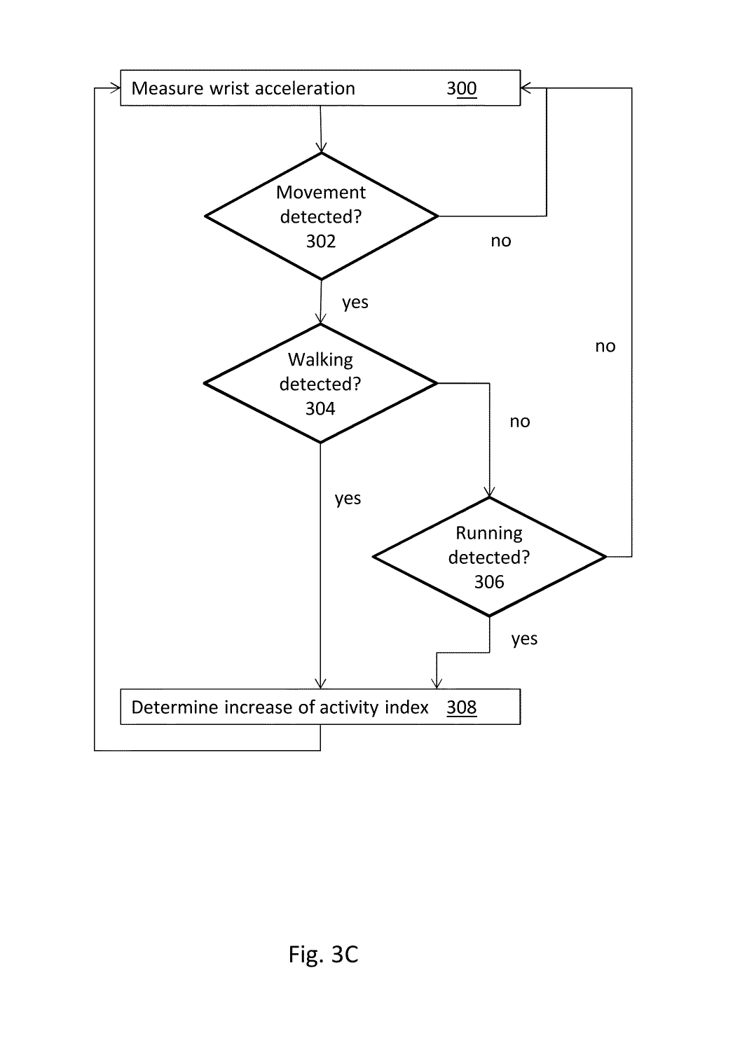

Explaining now in more detail a practical implementation of the automatic activity monitoring mode of the device, FIG. 3A shows a flow chart including main steps of the present method according to one embodiment. Activity monitoring in the automatic mode is started in step 31. This may occur by turning on the device or in response to a separate user action through the user interface. Then the device starts measuring acceleration continuously in step 32 (measurement continues preferably in the background although herein illustrated as a single step for simplicity). The acceleration signal is analyzed in step 33 in the data processing unit in order to detect cadence of the person. This is discussed in detail below. The acceleration signal is also used to calculate the average acceleration over a predefined time period. Then, if cadence is detected in the first place, it is checked in step 35 whether the cadence and average acceleration are within predefined cadence and acceleration ranges which are indicative of walking and/or running. In the negative case, the steps 32-35 are repeated. In the affirmative case, the cadence and average acceleration are used to compute a parameter which is descriptive of the intensity of the walking or running activity which is going on (step 36), and the activity index value is increased accordingly (step 37). Then, the process returns to step 32.

Cadence can be determined in the data processing unit by investigating the periodically repeating features, such as acceleration peaks, in the acceleration signal. According to one embodiment, the features being repeated are detected and their time stamps are recorded. The frequency and regularity, i.e. periodicity, of the features can be determined from the time stamps. If the frequency if too low or too high or the features do not occur regularly enough, the peaks do not represent cadence originating from walking or running. Predefined criteria for the allowed frequency and regularity ranges can be used. Regularity can be assessed for example by calculating correlation between intervals between successive time stamps. The correlation factor must exceed a predefined threshold correlation factor value. Such correlation analysis can be implemented in a wearable device efficiently in the time domain.

In an alternative embodiment, cadence is determined in the data processing unit using Fourier analysis for the acceleration data signal to determine its periodic components. High enough presence of periodic components in a predefined frequency range is an indication of walking or running. Discrete Fourier analysis can also be relatively efficiently implemented in a wearable device.

FIG. 3C illustrates one embodiment of the invention, starting with measurement of wrist acceleration in step 300. The processing unit is configured to first determine (step 302) whether said acceleration data exceeds a predefined magnitude threshold (e.g. the instant magnitude at the moment of measurement or the average magnitude over a measurement period). Only if said acceleration data exceeds the predefined magnitude threshold, the processing unit continues to determine (step 304) whether the acceleration data corresponds to walking-related movements of the person using a first method and/or first criteria of detecting frequency of motions (cadence) from the acceleration data. Only if the acceleration data corresponds to walking-related movements of the person, the activity index value is updated (step 308) based on the acceleration data. In a further embodiment, if the acceleration data does not correspond to walking-related movements, the unit may be configured to determine (step 306) if the acceleration data corresponds to running-related movements of the person using a second method and/or second criteria of detecting cadence. Again, if the acceleration data corresponds to the running-related movements of the person, the activity index value is updated (step 308) based on the acceleration data. If the magnitude criteria and neither of the walking or running criteria is met, the activity index value is not updated. The described embodiments help to optimize the battery consumption of the device. It is computationally a relatively lightweight process to determine whether there is such acceleration that may originate from walking or running. Further, since there is typically more walking than running during an ordinary persons day, it is advantageous to check the walking criteria before the running criteria.

According to one embodiment, the cadence detection process comprises two parts: calculation of cadence of slow motions c_{slow} and calculation of cadence of fast motions c_{fast}, c_{slow} typically having higher priority. This means that the process returns the value of c_{slow} if different from zero. If c_{slow} is zero, the value of c_{fast} is returned. The calculation of both c_{slow} and c_{fast} relies on detection of individual motions and checking if the motions repeat in cyclic manner. Otherwise, there is no cadence and a value is not returned at all. According to one embodiment the determination of c_{slow} utilizes convolution for the detection of cyclicality. c_{fast} may be implemented in a computationally more lightweight manner, using as the cyclicality criteria that n preceding time intervals between previous motions are within a predefined time window.

The slow motions referred to above can be in particular those corresponding to a first frequency (the frequency of walking), and the fast motions those corresponding to a second frequency (cadence) higher than that of the first frequency (the frequency of running).

FIG. 3B shows a preferred method in more detail, also including an optional autocalibration of acceleration-to-speed conversion carried in the method (dashed parts). The step 32 of FIG. 3A has been split into two sub-steps 32A and 32B, in which the acceleration is first measured in three dimensions to provide acceleration data separately for the three dimensions and then the total activity-related acceleration is computed based on the dimension-specific data. The activity related acceleration is obtained by computing the total magnitude of the acceleration vector, and optionally subtracting a gravity-based component from the acceleration data and/or filtering e.g. vibration- or noise-based components from the acceleration data.

The activity parameter determination step 36 has as well been split into to substeps 36A and 36B. In step 36A, the walking or running speed of the person is estimated based on the cadence and/or average acceleration data using a conversion function 39A (conversion function A) stored in the memory of the device. The conversion function 39A may be in the form of a mathematical formula, such as a piecewise linear function, or in table format, to mention some examples. The conversion function 39A uniquely binds average acceleration values to speed values. Preferably, the conversion function 39A has been determined taking the persons personal properties into account (such as running or walking style, affecting the average acceleration and/or cadence, and/or step length). For this purpose, an autocalibration method is described below, although manual calibration using user-input data is possible too. In step 36B, a relative intensity of the activity is determined based on the speed determined in step 36A. Again, a conversion function 39B (conversion function B) taking into account personal data of the user, is used. The personal data may comprise one or all of the following: age of the person, sex of the person, mass of the person, fitness index of the person, maximum oxygen intake value (VO_{2,max}) of the person. The conversion function may be a simple mathematical linear or non-linear formula taking the speed as input and using the personal data as weighing factors.

The autocalibration method referred to above for conversion function A utilizes a separate speed sensor, such as a satellite positioning sensor or foot pod, which gives a more reliable speed reading (“true” speed reading) than a purely wrist acceleration-based method, and is functionally connected to the present device. The connection may be a wired connection (e.g. a GPS receiver built-in in the device) or a wireless connection (e.g. a separate GPS receiver module or acceleration-based foot pod). If the device detects the presence of such sensor (step 38A), it uses a true speed reading provided by that sensor, for calibrating the conversion function 39A to reflect true dependency of speed on cadence and/or average acceleration. If the activity includes running and/or walking at practically all possible speeds, the whole conversion function becomes calibrated. The calibration can be done also piecewise, correcting only those parts of the conversion function that corresponds to the true speed readings available.

Autocalibration can be carried out when the device is in the first activity monitoring mode or in a second activity monitoring mode (e.g. a separate walking or running training mode specifically initiated by the user), which are discussed in more detail later. In particular if a satellite positioning sensor is used as a reference in the autocalibration, the latter option is preferred because it ensures that the reference data originates from pure walking or running and not for example from movement during a boat trip. Thus, the possibility for incorrect calibrations is decreased.

According to one embodiment, the conversion step 36A is based solely on average acceleration-to-speed conversion. An example of this kind of conversion is given later in this document.

As concerns the accuracy of speed (and optionally distance) determination, and therefore the activity index, the cadence determination step 33, autocalibration step 39A and cadence check step 35 play an important role. This is mainly because wrist is a difficult location to be used for speed determination, if particular at slow speeds, and an error at these phases may result in other motions than those relating to walking and/or running are being recorded, which is not the aim in the automatic activity measurement mode.

It should be noted that in the automatic activity monitoring mode in the core of the present invention, the device is preferably adapted to only detect and record activities that fall into the categories of walking and/or running. If there is any other kind of activity taking place, even if producing a measurable acceleration signal, it is either left unrecorded or recorded in another activity monitoring mode initiated by the user manually. The walking and/or running activity detected is transformed into an activity index increase using the characteristics of the acceleration. In this way, the most common activities typically forming a major portion of all additional metabolism on top of baseline metabolism, are recorded very accurately in the background and without a need for the user to separately put the device into a training mode.

There are three preferred (and also sufficient) data processing steps, which allow for detection of walking and/or running. The first step is the reliable determination of cadence, i.e., periodic features of the acceleration signal occurring with sufficient regularity (corresponding to regularity of wrist movement during walking and/or running). The second step is checking that the cadence is at an allowed frequency range (corresponding to frequency of wrist movement during walking and/or running). The third step is checking that the average acceleration calculated based on the measured acceleration data is at an allowed acceleration range (corresponding to possible wrist movement during walking and/or running). All these steps has been discussed above in detail, and can be carried out in practice in different ways and in various stages of data processing. According to a preferred embodiment, all these checks are carried out, making the detection very robust.

According to one embodiment, walking and running are detected using the same algorithm, such as the algorithm described above, by choosing the allowed cadence regularity, and/or cadence frequency and acceleration ranges to cover both walking and running.

According to another embodiment, only running is determined using the algorithm described above, and if the criteria for running are not met, a different algorithm for walking is used. The walking detection algorithm may be based for example in measuring acceleration in three dimensions to provide three-dimensional acceleration data, detecting characteristic features (corresponding to wrist motions) of these acceleration data, determining correlation factors between the characteristic features in all the three dimensions. The correlation factors define three-dimensional correlation vectors, whereby the determination of whether the motion measured corresponds to walking can be evaluated based on direction and magnitude of the correlation vectors (i.e. spatial location of the dimension-specific correlation factors). The correlation factors are calculated for each characteristic feature detected such that data values at predefined points of the feature are multiplied with data values at corresponding points of a previous detected feature. The multiplication results can then be summed to obtain a correlation factor depicting correlation between the two features. The correlation factors can then be saved as a time series for further use.

According to one embodiment, the activity index represents a relative intensity of training. The higher the physical intensity of the training, the faster the activity index is increased. Higher intensity of running compared to walking is reflected in higher cadence and higher average acceleration. The processing unit is adapted to increase the activity index based on one or both of them more rapidly during running than during walking.

The activity index can be dedicated to running and/or walking only, i.e., it is not updated if any other physical activity is detected. The activity index can also be common to all activities the device is capable of detecting, i.e., is updated also in other activity monitoring modes than the running and/or walking mode.

Also other quantities than the activity index can be computed based on intermediate values of the processing, such as cadence, speed or average wrist acceleration, or the end value, i.e. the activity index. For example, cadence can be used to compute the number of steps taken by the person if multiplied by time. Similarly, speed can be used to compute the distance travelled. The activity index can be used to compute the energy consumption, the training effect, the peak training effect, or the recovery time needed.

According to one embodiment, in addition to the first automatic activity monitoring mode discussed above, there are one or more second activity monitoring modes defined in the device, for example for gym training, swimming, cycling and/or various team sports. Such second activity monitoring modes can be started and stopped through the user interface of the device. Such manually started mode may override the automatic mode, i.e., the activity index is updated using the update criteria of the second mode instead of the first mode, or instead another activity index is updated. The second activity monitoring mode(s) can use also other sensors than the built-in acceleration sensor, for example a heartbeat sensor, satellite positioning sensor, bike pod or foot pod. The other sensor(s) may be built-in or in wireless communication with the present device.

The first activity monitoring mode may be operational whenever the present device is switched on, or according to a calendar set by the user, or upon user input, to mention some examples. Even if programmed to require user input to start/stop, the first activity monitoring mode distinguishes from the one or more second activity monitoring modes in that the update criteria of the activity index are bound to the detection of walking or running, whereas in the other modes the monitoring are typically not depending on such detection, as different criteria are used. Preferably, there are at least two sports-type specific second modes that are launched through the user interface of the device by selecting the particular sports the user is going to exercise, after which all data provided by the acceleration or other sensor(s) is interpreted to belong to that sports exercise until the user terminates the mode through the user interface.

In one embodiment, all other movement activity measured by the acceleration sensor than that recorded in the first activity monitoring mode and optionally in the one or more second activity monitoring modes, are determined to be part of baseline activity, i.e. is not specifically recorded, or, if recorded, as a separate activity index different than that used in the first and/or second activity monitoring mode(s).

In one embodiment, in the automatic activity monitoring mode, if one or all of the abovementioned checks (cadence regularity and frequency and acceleration strength) fail, while the overall intensity of acceleration remains at a predefined level for a predefined period, the device suggests the user through its user interface to manually start a second activity monitoring mode corresponding to the activity taking place.

### EXAMPLE

Acceleration values (aₓ, a_{y} and a_{z} in three orthogonal directions x, y, z are measured using acceleration sensors. The total magnitude of the acceleration vector is a₁:

a₁=\sqrt{aₓ²+a_{y}²+a_{z}²}.

Gravity g₀ is subtracted from total acceleration, yielding movement-related acceleration a₂:

a₂=a₁−g₀.

A plurality of movement-related acceleration values a₂ are measured over a time period Δt and used to calculate average movement acceleration aₐᵥₑ

$${a}_{ave} = \frac{\sum {a}_{2} ( t )}{Δ t} .$$

Then, the average acceleration aₐᵥₑ is converted to running speed v_{running} using a piecewise linear function f_{mapping( )}, also applying a cadence check function f_{cadenceCheck( )}, which checks that aₐᵥₑ and cadence detected from the acceleration signal are at allowed ranges:

v_{running}=f_{cadenceCheck}(cadence,aₐᵥₑ)f_{mapping}(aₐᵥₑ).

The mapping funtion f_{mapping( )} can have a predefined form, but its form can also be changed using reference measurements to adapt the function to individual running characteristics of the person.

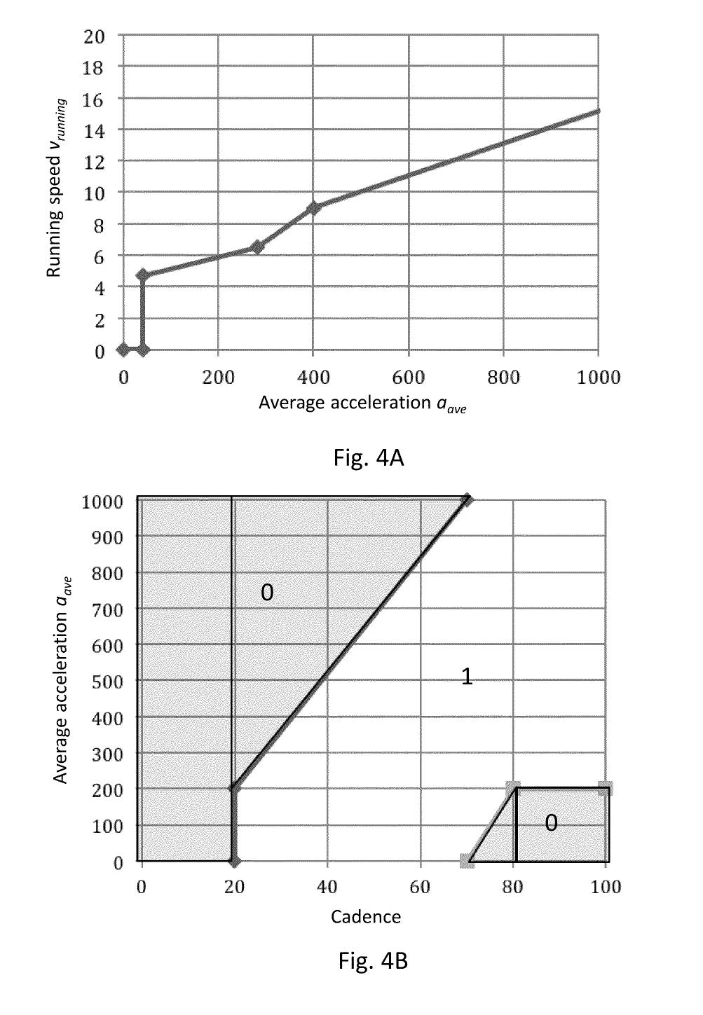

According to one embodiment, the adaptation of the mapping function is carried out as an autocalibration without user involvement. Autocalibration may for example take place if the user switches on a speed measurement module such as a satellite positioning sensor or footpod sensor yielding the true speed of the person. The present device receives the speed data obtained using such sensors, and utilizes the speed information for updating the piecewise linear function to reflect the correct relation between aₐᵥₑ and the measured reference speed. The function is then saved for further speed determination as described above. The device may also itself command a speed measurement module to perform the autocalibration. FIG. 4A shows an example of a piecewise linear mapping function.

The value of the cadence check function f_{cadenceCheck( )} is either 1 or 0, depending on whether both cadence and aₐᵥₑ are within predefined ranges or not. FIG. 4B shows an example of a cadence check function. In the central portion, if cadence and average acceleration are lying between the thick lines, the measurement can be determined to originate from running or walking, and the function takes the value 1. Outside this zone, the value is 0, rendering the value of v_{running} also 0.

It has been found in practical tests, that the device and method as herein described provides results that are in good correlation with reality. Among twenty test persons, the method produced only a 5% average error in distance travelled (computed from speed), compared to a distance determined using a foot pod, even if using a predefined cadence/acceleration-to-speed conversion. The standard deviation was nearly 30%, but autocalibration of the cadence/acceleration-to-speed conversion is expected to reduce this figure significantly, because individual variations in motion trajectories are then being taken into account.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures or characteristics may be combined in any suitable manner in one or more embodiments. In the description numerous specific details are provided to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

1. A device for monitoring physical activity of a person, comprising
an acceleration sensor for providing acceleration data corresponding to movements of the person,
a memory unit capable of storing an activity index value describing a cumulative physical activity of the person, and
data processing unit capable of being operated in at least a first activity monitoring mode in which the data processing unit is adapted to update said activity index value based on said acceleration data,
wherein in said first activity monitoring mode the processing unit is configured to determine if, and to update the activity index value only if, the acceleration data corresponds to walking- and/or running-related movements of the person.

2. The device according to claim 1, wherein the processing unit is configured to determine if the acceleration data corresponds to walking- and/or running-related movements of the person by analyzing the periodicity of the acceleration data.

3. The device according to claim 1, wherein the processing unit is configured to determine if the acceleration data corresponds to walking- and/or running-related movements of the person by analyzing the average magnitude of the acceleration data.

4. The device according to claim 1, wherein in said first activity monitoring mode, the data processing unit in configured to
detect periodic motions from the acceleration data,
determine frequency of the periodic motions,
determine whether the frequency of the periodic motions is at a predefined frequency range, and
update the activity index value using said frequency of periodic motions only if the frequency of the periodic motions is at said predefined frequency range.

5. The device according to claim 1, wherein in said first activity monitoring mode, the data processing unit is configured to
determine average acceleration based on the acceleration data,
determine whether the average acceleration is within a predefined acceleration range, and
update the activity index using said average acceleration only if the average acceleration is within said predefined acceleration range.

6. The device according claim 1, wherein in said first activity monitoring mode the processing unit is configured to
determine average acceleration based on the acceleration data,
convert the average acceleration determined to average speed of the person using a conversion function stored in the memory of the device, and
use the average speed for updating the activity index value.

7. The device according to claim 6, further comprising means for receiving speed data of the person from a speed sensor and to adapt properties of the conversion function in the memory of the device so as to reflect the correct dependence between the average acceleration and said speed data.

8. The device according to claim 1, wherein the processing unit is additionally configured to use at least one of the following parameters storable in the memory of the device for updating the activity index value: age of the person, sex of the person, mass of the person, fitness index of the person, maximum oxygen intake value (VO_{2,max}) of the person.

9. The device according to claim 1, wherein the processing unit is capable of being operated in at least one second activity monitoring mode in which the processing unit is configured to update the activity index value using different update criteria.

10. The device according to claim 9, further comprising user interface means for allowing the person to switch between the first and second activity monitoring modes of the data processing unit.

11. The device according to claim 1, wherein it comprises means for mounting the device on the wrist of the person.

12. The device according to claim 1, further comprising at least one other sensor than the acceleration sensor or means for wirelessly receiving data form such other sensor, and the data processing unit being adapted to operate, in response to user action through user interface means of the device, in a second activity monitoring mode utilizing data form said other sensor for monitoring the activity of the person.

13. The device according to claim 1, wherein in said first activity monitoring mode the processing unit is configured
to determine whether said acceleration data exceeds a predefined magnitude threshold,
only if said acceleration data exceeds said predefined magnitude threshold, to determine whether the acceleration data corresponds to walking-related movements of the person, and
if the acceleration data corresponds to said walking-related movements of the person, to update the activity index value based on the acceleration data.

14. The device according to claim 13, wherein in said first activity monitoring mode the processing unit is further configured
if the acceleration data does not correspond to said walking-related movements of the person, to determine if the acceleration data corresponds to running-related movements of the person, and
if the acceleration data corresponds to said running-related movements of the person, to update the activity index value based on the acceleration data.

15. A device for monitoring physical activity of a person, comprising
an acceleration sensor for providing acceleration data corresponding to movements of the person,
a memory unit capable of storing an activity index value describing a cumulative physical activity of the person, and
a data processing unit adapted to update said activity index value based on said acceleration data according to update criteria,
wherein
the data processing unit is capable of being operated in at least a first activity monitoring mode and a second activity monitoring mode, and
the data processing unit is configured to use different update criteria in said first activity monitoring mode than in said second activity monitoring mode.

16. A device for monitoring physical activity of a person, comprising
an acceleration sensor for providing acceleration data corresponding to movements of the person, and
a data processing unit for analyzing the acceleration data for computing an activity index describing a cumulative activity of the person, the data processing unit comprising means for detecting periodic motions from the acceleration data, means for determining frequency of the periodic motions, and means for determining whether the frequency of the periodic motions is within a predefined frequency range,
wherein said data processing unit further comprises
means for determining average acceleration based on the acceleration data,
means for determining whether the average acceleration is within a predefined acceleration range, and
means for updating the activity index using said frequency of periodic motions and average acceleration if the frequency of the periodic motions and the average acceleration are within said predefined ranges.

17. A method for monitoring physical activity of a person in a wearable device, comprising
providing acceleration data corresponding to wrist movements of the person, and
analyzing the acceleration data unit in a first activity monitoring mode of the wearable device in order to derive an activity index corresponding to the intensity of the physical activity,
wherein the acceleration data is analyzed in said first activity monitoring by detecting features of the acceleration data that correspond to cadence of walking and/or running, and if such features are found, updating the activity index using said features.

18. The method according to claim 17, wherein said features comprise periodic acceleration peaks in the acceleration data being repeated at a predefined frequency range, and/or total average acceleration determined from the acceleration data over a predefined time period at a predefined acceleration range.
